(19) United States
(12) Patent Application Publication
(10) Pub. No.: US 2016/0041531 A1
(43) Pub. Date: Feb. 11, 2016

(54) BIOFEEDBACK WATCHES

(71) Applicant: Quanttus, Inc., Cambridge, MA (US)

(72) Inventors: Scott Mackie, New Haven, CT (US); Chris Loughnane, Brookline, MA (US); Jordan Rice, Portland, OR (US); David Da He, Cambridge, MA (US); Brian Schiffer, Brighton, MA (US); Richard Robehr Bijani, Cambridge, MA (US); Gen Suzuki, Kanagawa (JP); Matthew Brown, Somerville, MA (US); Steve Lowe, Seattle, WA (US); Kate Richmond, Seattle, WA (US); Jesse Allum, Seattle, WA (US); Ray Nicoli, Seattle, WA (US); Bret Richmond, Seattle, WA (US); Eric Healy, Lynnwood, WA (US); Pam Darvirris, Seattle, WA (US); Stephen Kreiger, Kent, WA (US); Gregory Wolos, Cambridge, MA (US); Narissa Chang, Somerville, MA (US); Zebrick Roach, Arlington, MA (US)

(21) Appl. No.: 14/819,976

(22) Filed: Aug. 6, 2015

Related U.S. Application Data
(60) Provisional application No. 62/033,935, filed on Aug. 6, 2014.

(51) Int. Cl. G04G 21/02; G04G 19/00; A61B 5/00; A61B 5/11; A61B 5/1455; G04G 9/00; A61B 5/0205

(52) U.S. Cl. CPC G04G 21/025; G04G 9/0064; G04G 19/00; A61B 5/02055; A61B 5/1102; A61B 5/14551; A61B 5/681; A61B 5/02125

(57) ABSTRACT

A watch comprising a deadfront window; a first OLED display disposed beneath the deadfront window such that the first OLED display can be observed through the deadfront window when the first OLED display is active; a movement for tracking time, the movement having a first portion that resides beneath the deadfront window and a second portion that protrudes through the deadfront window; and time-indicating members disposed above the deadfront window, the time indicating members being secured to the second portion of the movement.

### CLAIM OF PRIORITY

This application claims priority to U.S. Provisional Patent Application Ser. No. 62/033,935, filed on Aug. 6, 2014, the entire contents of which are hereby incorporated by reference.

### TECHNICAL FIELD

This document relates to biofeedback watches.

### BACKGROUND

The use of wristwatches for telling time has been around for a long time. More recently, wristbands have been used for collecting certain health-related data.

### SUMMARY

In one aspect, a watch includes a deadfront window and a first OLED display disposed beneath the deadfront window. The first OLED display is disposed beneath the deadfront window such that the first OLED display can be observed through the deadfront window when the first OLED display is active. The watch also includes a movement for tracking time. The movement has a first portion that resides beneath the deadfront window and a second portion that protrudes through the deadfront window. The watch also includes time-indicating members disposed above the deadfront window. The time indicating members are secured to the second portion of the movement.

Implementations can include one or more of the following features.

In some implementations, the deadfront window is a semi-transparent window that allows light to pass through when a nearby light source is active. The deadfront window is substantially opaque in the absence of a nearby active light source.

In some implementations, the watch is a biofeedback watch.

In some implementations, the first OLED display is hidden beneath the deadfront window when the first OLED display is inactive.

In some implementations, the watch also includes a second OLED display. The first OLED display resides beneath a first portion of the deadfront window, and the second OLED display resides beneath a second portion of the deadfront window.

In some implementations, the portion of the movement that protrudes through the deadfront window includes an extender that is secured to a post of the movement.

In some implementations, the first OLED display has a resolution of 128×32 pixels.

In some implementations, the first OLED display is configured to emit colored light.

In some implementations, the first OLED display is a passive-matrix OLED display.

In some implementations, the watch also includes a processor that is electrically connected to the first OLED display. The processor is configured to generate data related to one or more biometric measurements. The processor is also configured to cause the first OLED display to display information related to one or more of the biometric measurements.

In some implementations, the biometric measurements include one or more of heart rate, pulse transit time, stroke volume, systolic and diastolic blood pressure, and cardiac output.

In some implementations, the first OLED display does not display a time of day.

In some implementations, the watch also includes a case that contains the first OLED display and the movement.

In some implementations, the watch has a maximum thickness, as measured from a bottom surface of the case to a top surface of a bezel that is secured to the case, of less than 8.80 mm.

In some implementations, the case has a diameter of less than 38 mm.

In some implementations, the watch also includes an optical sensor and an LED that are each disposed within the case.

In some implementations, the watch also includes an insert disposed in an aperture formed by a bottom wall of the case. The insert has a first opening aligned with the optical sensor and a second opening aligned with the LED.

In some implementations, the case is a one-piece case.

In some implementations, the watch also includes a printed circuit board electrically connected to the first OLED display and the movement. The printed circuit board is configured to provide power to the first OLED display, the movement, and the processor.

In some implementations, the printed circuit board is configured to provide power to the movement via a contact spring.

In some implementations, the printed circuit board is electrically connected to a watch battery.

In some implementations, the watch has a single power source.

In some implementations, the single power source is not directly connected to the movement.

In another aspect, a watch includes a one-piece case. The one-piece case has a bottom wall and an outer wall extending from a circumferential region of the bottom wall. The bottom wall forms an aperture. The watch also includes a dial secured to the case. The watch also includes a movement for tracking time. The movement has a first portion that resides beneath the dial and a second portion that protrudes through the dial.

The watch also includes time-indicating members disposed above the dial. The time indicating members are secured to the second portion of the movement. The watch also includes an insert disposed in the aperture formed by the bottom wall of the one-piece case. The insert has a first opening aligned with an optical sensor disposed within the case. The insert also has a second opening aligned with an LED disposed within the case. The insert also has a wall that separates the first opening from the second opening.

Implementations can include one or more of the following features.

In some implementations, the watch is a biofeedback watch.

In some implementations, the watch also includes a window that resides in the first opening and a lens that resides in the second opening.

In some implementations, the insert is made of one contiguous piece of material. In some implementations, the insert has a third opening configured to align with a second LED disposed within the case. The wall separates the first opening from the third opening.

In some implementations, the wall is a first ring-shaped member.

In some implementations, the insert has a second ring-shaped member concentrically disposed around the first ring-shaped member.

In some implementations, the first ring-shaped member defines the first opening and the first and second ring-shaped members cooperate to define the second and third openings.

In some implementations, the insert has segments that extend between the first and second ring-shaped members and separate the second opening from the third opening.

In some implementations, the wall prevents light emitted from the LED from reaching the optical sensor until the emitted light passes through the second opening.

In some implementations, the watch also includes a display disposed beneath the dial. The watch also includes a processor that is electrically connected to the display. The processor is configured to generate data related to one or more biometric measurements. The processor is also configured to cause the display to display information related to one or more of the biometric measurements. The watch also includes a printed circuit board electrically connected to the display, the processor, and the movement. The printed circuit board is configured to provide power to the display, the processor, and the movement.

In some implementations, the biometric measurements include one or more of heart rate, pulse transit time, stroke volume, systolic and diastolic blood pressure, and cardiac output.

In some implementations, the printed circuit board is configured to provide power to the movement via a contact spring.

In some implementations, the printed circuit board is electrically connected to a watch battery.

In some implementations, the watch has a single power source.

In some implementations, the single power source is not directly connected to the movement.

In another aspect, a watch includes a dial. The watch also includes a movement for tracking time. The movement has a first portion that resides beneath the dial and a second portion that protrudes through the dial. The watch also includes time-indicating members disposed above the dial. The time indicating members are secured to the second portion of the movement. The watch also includes a display disposed beneath the dial. The watch also includes a printed circuit board electrically connected to the display and the movement. The printed circuit board is configured to provide power to the display and the movement.

Implementations can include one or more of the following features.

In some implementations, the watch is a biofeedback watch.

In some implementations, the watch also includes a processor that is electrically connected to the printed circuit board and the display. The processor is configured to generate data related to one or more biometric measurements. The processor is also configured to cause the display to display information related to one or more of the biometric measurements.

In some implementations, the biometric measurements include heart rate, pulse transit time and stroke volume, systolic and diastolic blood pressure, and cardiac output.

In some implementations, the printed circuit board is configured to provide power to the processor.

In some implementations, the printed circuit board is configured to provide power to the movement via a contact spring.

In some implementations, the printed circuit board is electrically connected to the watch battery.

In some implementations, the watch has a maximum thickness, as measured from a bottom surface of a case of the watch to a top surface of a bezel that is secured to the case, of 8.80 mm.

In some implementations, a case of the watch has a diameter of less than 38 mm.

In some implementations, the watch has a single power source.

In some implementations, the single power source is electrically connected to the printed circuit board via a first connection and the movement is connected to the printed circuit board via the contact spring.

In some implementations, the single power source is positioned adjacent to the movement.

In some implementations, the watch also includes an optical sensor and an LED. The single power source is configured to provide power to the printed circuit board, the processor, the display, the movement, the optical sensor, and the LED.

In some implementations, the display does not display a time of day.

Implementations can include one or more of the following advantages.

In some implementations, the OLED display resides beneath a deadfront window. This configuration is advantageous because it allows the biofeedback watch to take on different appearances depending on how the biofeedback watch is being used at the time. For example, if the biofeedback watch is being used to view biofeedback information, the OLED display is active and visible through the deadfront window, giving the biofeedback watch an active appearance. However, if the biofeedback watch is not being used to view biofeedback information at the time, and instead is being used simply to tell time, the deadfront window hides the internal components of the biofeedback watch (e.g., the OLED display), giving the biofeedback watch the appearance of a traditional analog watch. The high-resolution OLED display itself is advantageous because it is capable of displaying complex images.

In some implementations, the biofeedback watch has a one-piece (e.g., monoblock) case. A one-piece case is advantageous because it limits the positional variances of the components of the biofeedback watch. For instance, if a case is made up of multiple sections, the positions of the sections relative to one another can vary. The more sections there are, the greater the total variance is between the sections. For example, it is advantageous for the LEDs and the optical sensor to precisely line up with the sensor assembly insert so that the optical sensor can make accurate measurements. The one-piece case eliminates a number of positional variances that would otherwise be present in a traditional watch that has a separate bottom wall (e.g., a removable bottom plate). The one-piece case can, for example, be a monoblock component defining an aperture in its rear surface for receiving the sensor assembly insert. The sensor assembly insert has openings configured to align with the LEDs and the optical sensor. The sensor assembly insert is designed to fit securely within the aperture of the case to ensure the LEDs and optical sensor properly align with the openings in the sensor assembly insert. Due to the one-piece design of the case, it is not necessary to assemble other components that might cause the openings to become misaligned. The openings are formed by a one-piece sensor assembly insert, reducing or eliminating positional variance between the openings that may otherwise result if the openings were formed by separate components.

In some implementations, the sensor assembly has a first opening configured to align with the optical sensor and second openings configured to align with the LEDs. An inner wall and an outer wall form the openings and separate the LEDs from the optical sensor. The optical sensor can be configured to obtain photoplethysmographic (PPG) data. The LEDs can illuminate the skin of a user with light, and the optical sensor can measure the amount of light transmitted or reflected off of the skin. By providing walls that separate the LEDs from the optical sensor, light emitted from the LEDs is prevented from reaching the optical sensor before it is first illuminated on the skin of the user. This configuration can, therefore, increase the accuracy of PPG data collected and thus increase the accuracy with which certain vital signs of the wearer are determined.

In some implementations, the movement does not have its own independent battery, and instead, the movement is electrically connected to the printed circuit board, which is electrically connected to a primary watch battery. As such, the biofeedback watch can contain a single battery. This configuration is advantageous because it reduces the number of components in the biofeedback watch and allows the biofeedback watch to have a thinner overall profile, giving it the appearance of a traditional analog watch.

Other aspects, features, and advantages of the invention will be apparent from the description and drawings, and from the claims.

### DESCRIPTION OF DRAWINGS

FIG. 1 is a perspective view of a body of a biofeedback watch.

FIG. 2 is a perspective view of the watch body with a crystal, indicator ring, movement, and hands removed to expose a dial.

FIG. 3 is a perspective view of the watch body with the dial removed, exposing two OLED displays.

FIG. 4 is a perspective cross-sectional view of the watch body showing a battery and a movement of the watch.

FIG. 5 is a perspective cross-sectional view of the watch body showing a battery used to power the movement of the watch.

FIG. 6 is a perspective cross-sectional view of the watch body showing a printed circuit board assembly.

FIG. 7 is a perspective cross-sectional view of the watch body showing a USB contacts housing.

FIG. 8 is a bottom view of the watch body showing a sensor assembly insert and the USB contacts housing.

FIG. 9 is a perspective cross-sectional view of the watch body.

FIG. 10 is a perspective view of the sensor assembly insert of the watch body.

FIG. 11 is a perspective view of a charging dock for the biofeedback watch.

FIG. 12 shows a wristband connected to the watch body to form a fully assembled biofeedback watch.

FIGS. 13a-c are a series of screenshots of the biofeedback watch of FIG. 12 during an example use of the biofeedback watch.

FIG. 14 shows portions of another biofeedback watch body in which a movement is electrically connected to a printed circuit board of the watch body.

FIG. 15 shows portions of another biofeedback watch body in which an I-ring creates a seal between the crystal and the bezel of the watch.

FIG. 16 shows another sensor assembly insert of the watch body.

FIGS. 17a-b show portions of another biofeedback watch body in which the external sensor is a ring disposed inside the case.

FIGS. 18a-b show portions of another biofeedback watch body in which an ambient light sensor and ultraviolet sensor are electrically connected to the printed circuit board.

### DETAILED DESCRIPTION

This document describes biofeedback watches that can collect motioncardiogram (MoCG) data (which is related to ballistocardiogram (BCG) data) and photoplethysmographic (PPG) data and, in some cases, perform various biometric measurements (e.g., blood pressure, respiration rate, blood oxygen level, stroke volume, cardiac output, and temperature) based on the MoCG data and the PPG data. MoCG is a pulsatile motion signal of the body measurable, for example, by a motion sensor such as an accelerometer. The pulsatile motion signal results from a mechanical motion of portions of the body that occurs in response to blood being pumped during a heartbeat. This motion is a mechanical reaction of the body to the internal flow of blood and is externally measurable. The MoCG signal therefore corresponds to, but is delayed from, the heartbeat.

PPG is data optically obtained via a plethysmogram, a volumetric measurement of the vasculature. PPG can be obtained using an optical device which illuminates the skin and measures changes in light absorption. With each cardiac cycle the heart pumps blood resulting in a pressure pulse wave within the vasculature. This causes time-varying changes in the volume of the vasculature. The changes can be detected, for example, by illuminating the skin with light from a light-emitting diode (LED) and then measuring the amount of light either transmitted or reflected to a detector such as a photodiode. Each cardiac cycle is therefore represented as a pattern of crests and troughs. The shape of the PPG waveform differs from subject to subject.

Described herein are watches (e.g., a biofeedback watches) that can collect MoCG and PPG data and perform biometric measurements based on the collected MoCG and PPG data. The biometric measurements can be used for monitoring health related parameters, as well as in diagnosing conditions and predicting an onset of such conditions. In some cases, the biofeedback watch has an analog movement with hands positioned above a deadfront window and a high-resolution OLED display positioned beneath the deadfront window. The OLED display, when activated, is visible through the deadfront window and can be used to display information related to biometric measurements. When the biofeedback watch is not displaying information related to the biofeedback functions described above, it has the appearance of a traditional analog watch because the deadfront window hides underlying components and allows the user to clearly see only the hands positioned above the deadfront window. In certain cases, the movement does not include a separate battery. Rather, the biofeedback watch can contain a single battery, reducing the total number of components in the biofeedback watch. This can allow the biofeedback watch to be thinner overall, further giving it the appearance of a traditional watch.

In some cases, the biofeedback watch has a one-piece case, a one-piece sensor assembly insert, and an LED and optical sensor that are positioned to along with openings in the sensor assembly insert. The sensor assembly insert is designed to fit securely within an aperture of the case. Due to the one-piece design of the case and the sensor assembly insert, it is not necessary to assemble other components that might cause the openings to become misaligned with the LED and the optical sensor.

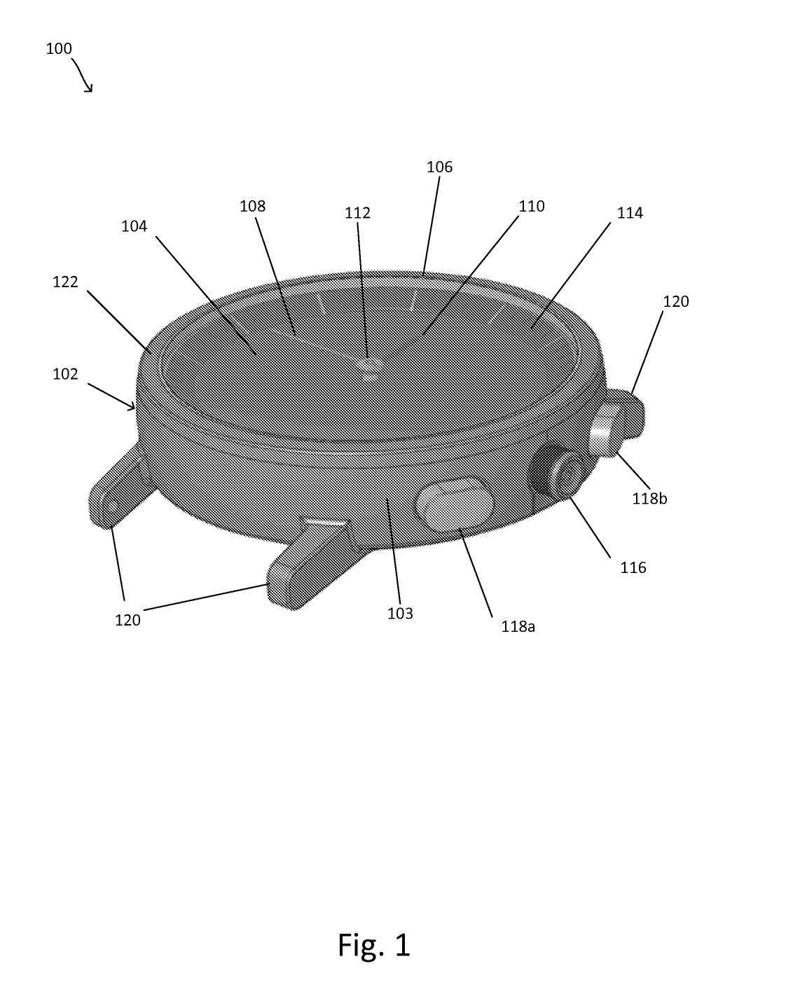

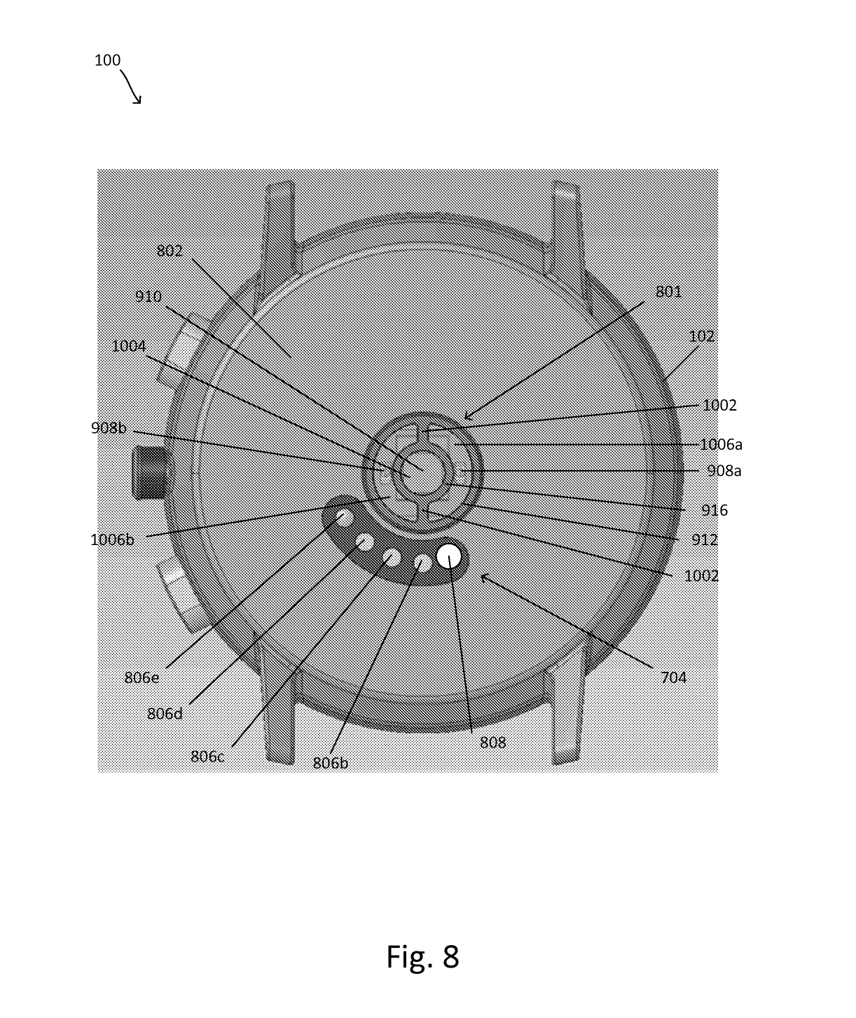

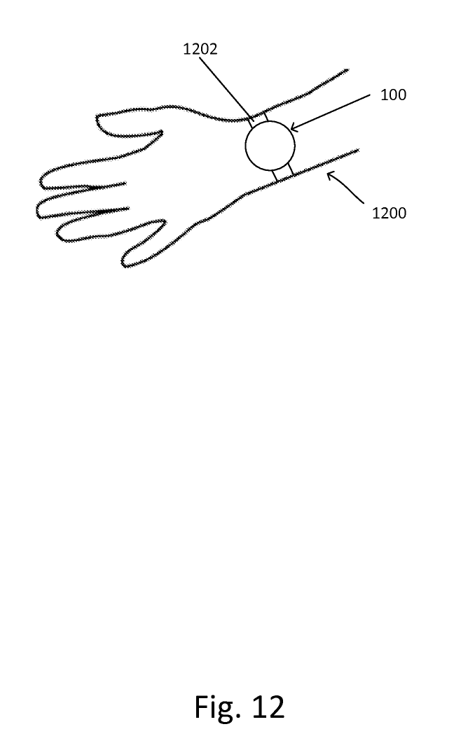

FIG. 1 shows a perspective view of a body 100 of a biofeedback watch. A wristband 1202 (shown in FIG. 12) is typically attached to the watch body 100 during use, but the wristband 1202 has been omitted from FIG. 1 and many subsequent figures for simplicity. As shown, when the biofeedback watch is not performing the biofeedback functions described above, it has the appearance of a traditional analog watch. The body 100 includes a case 102 that has a bottom wall 802 (shown in FIG. 8) and an outer wall 103. The case 102 is configured to hold the internal components of the biofeedback watch. The case 102 is formed from a single, contiguous piece of material such that the case 102 has a monoblock structure. As a result, the bottom wall 802 of the case 102 is not removable from the rest of the case 102. The case 102 also includes lugs 120 for connecting a strap or the wristband 1202 to the case 102.

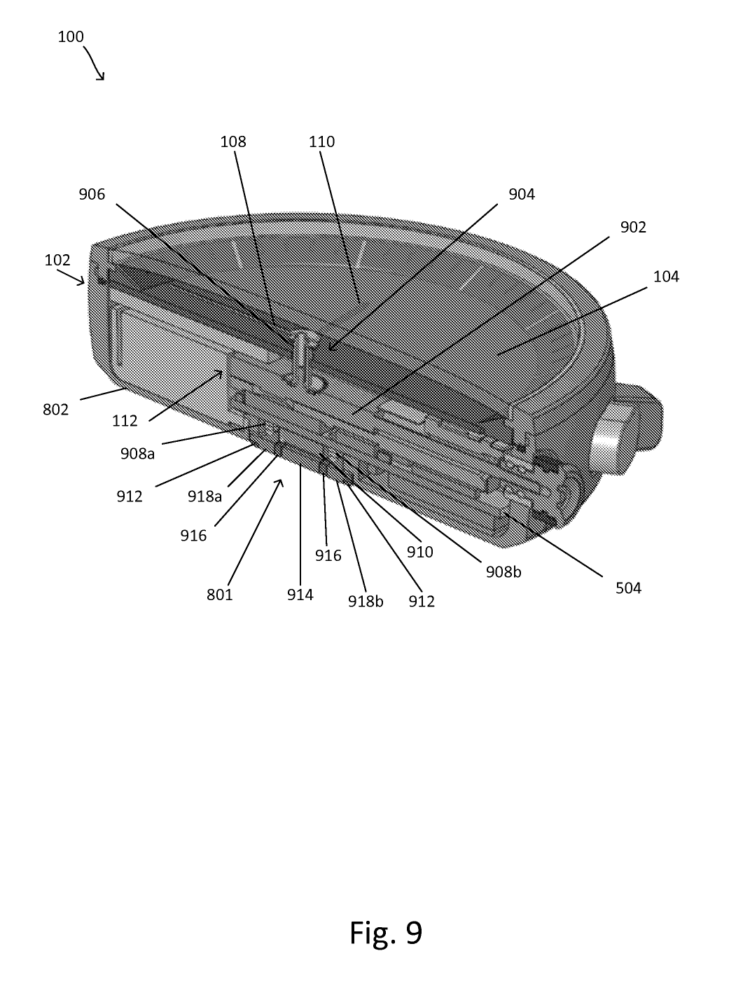

A dial 104 resides in the case 102 beneath time-indicating members (e.g., a minute hand 108 and an hour hand 110). The hands 108, 110 are attached to a movement 112. The movement 112 controls the timekeeping functions of the watch. The movement 112 has a first portion 902 (shown in FIG. 9) that resides beneath the dial 104 and a second portion 904 (shown in FIG. 9) (e.g., a post) that protrudes through the dial 104. The hands 108, 110 are attached to the second portion 904 of the movement 112. The second portion 904 of the movement 112 rotates about a central axis, causing the hands 108, 110 of the biofeedback watch to rotate. An indicator ring 114 that includes markings is positioned around the circumference of the dial 104. The movement 112 causes the hands 108,110 to point to markings on the indicator ring 114 to indicate a time of day.

The dial 104 is a deadfront window. A deadfront window is a semi-transparent window. When a nearby light source is active, the deadfront window allows light to pass through. However, in the absence of a nearby, active light source, the deadfront window is substantially opaque. The deadfront window is typically made of a material having deadfront characteristics. The deadfront window, as described below, allows the biofeedback watch to take on different appearances depending on how the biofeedback watch is being used at the time.

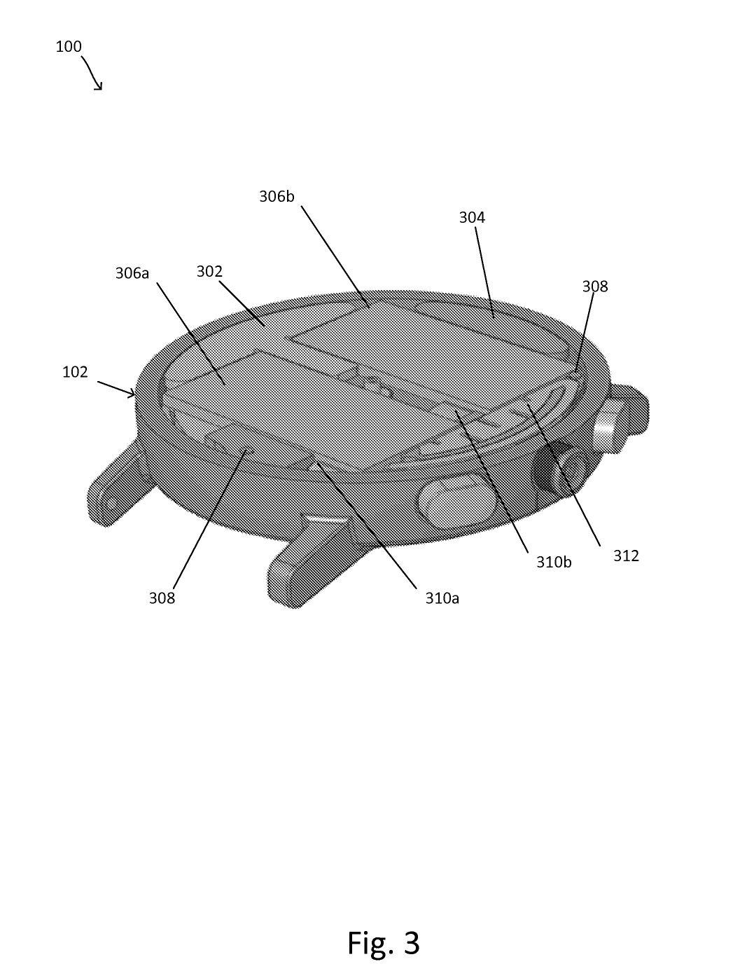

The dial 104 includes a transparent substrate and two layers of ink pigments that give the transparent substrate deadfront characteristics. The transparent substrate can be formed of any of a number of materials, such as glass, acrylic, polycarbonate, mineral glass, Gorilla Glass™, synthetic sapphire, or any other suitable material. The first ink pigment layer is substantially opaque and is deposited over the top surface of the dial 104 except for the regions of the dial 104 that align with the OLED displays 306a, 306b (shown in FIG. 3). The second ink pigment layer is substantially transmissive and is deposited over the entire top surface of the dial 104 on top of the first ink pigment layer. As such, the regions of the dial 104 that align with the OLED displays 306a, 306b allow more light to pass through than the other regions of the dial 104.

Referring to FIG. 3, two OLED displays 306a, 306b reside beneath the deadfront window. The OLED displays 306a, 306b emit light only when they are active. When the OLED displays 306a, 306b are active, the majority of the light passes through the deadfront window, and the display can be easily seen by a user. However, when the OLED displays 306a, 306b are inactive, they remain hidden or substantially hidden beneath the deadfront window. As such, the user cannot easily see the OLED displays 306a, 306b when the OLED displays 306a, 306b are inactive, and the dial 104 has the appearance of a traditional analog watch face.

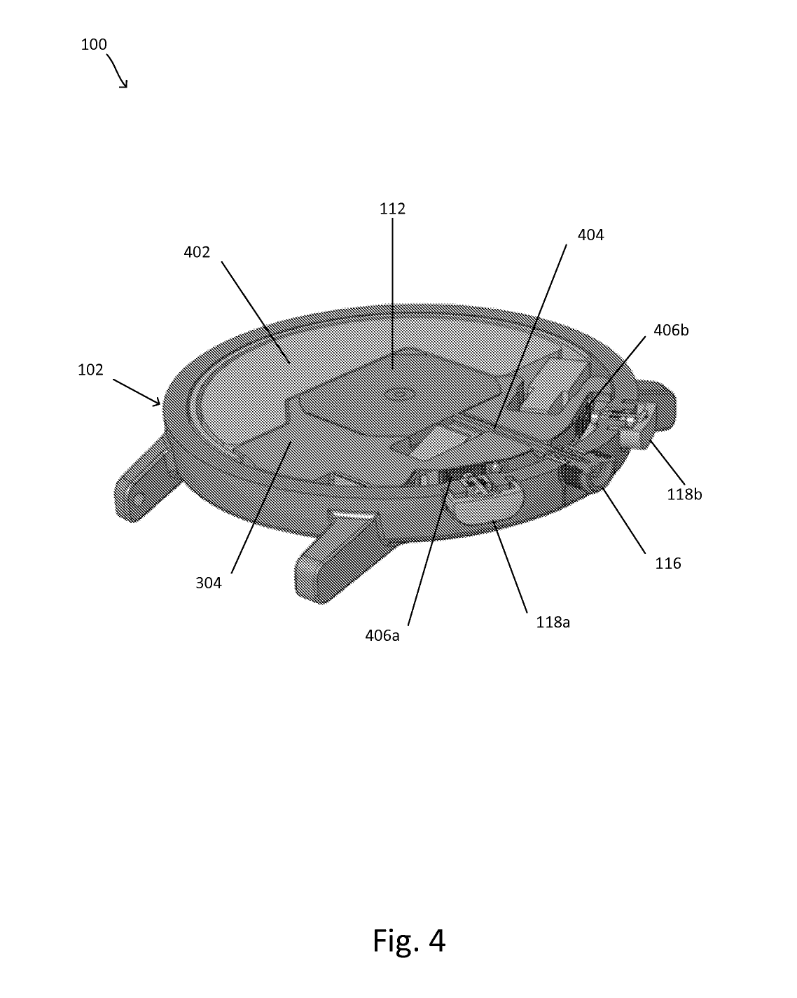

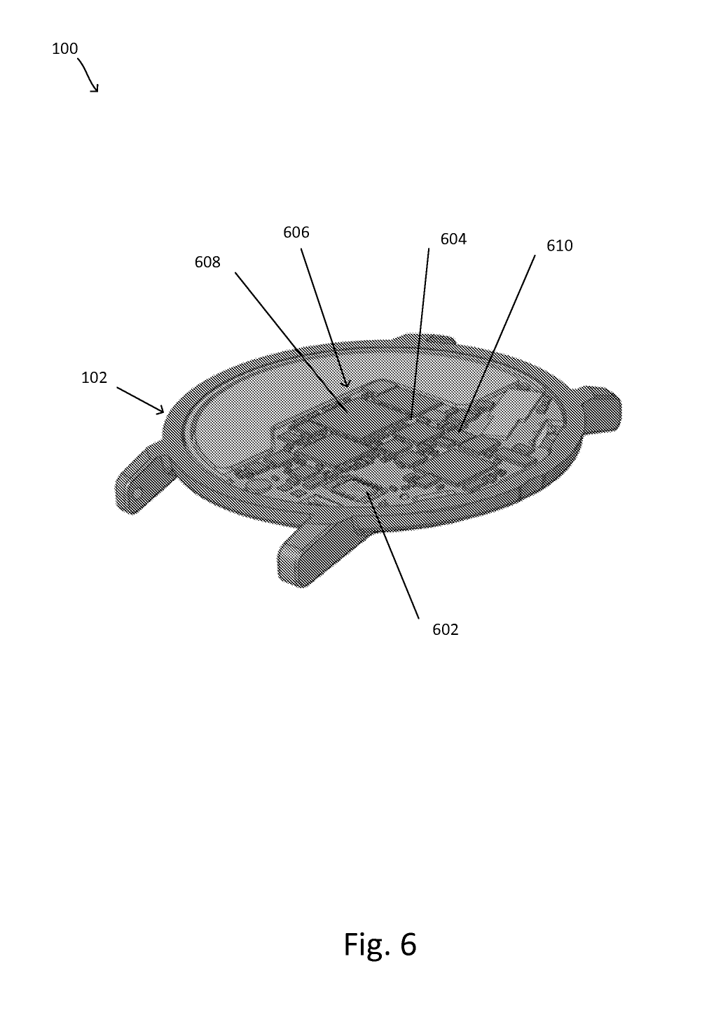

As shown in FIGS. 1 and 4, the case 102 includes apertures for accepting buttons 118a, 118b. The buttons 118a, 118b can communicate with a processor 608 (shown in FIG. 6) and cause the processor 608 to control the internal components of the biofeedback watch, as described in more detail below. The case 102 also includes an aperture for accepting a crown 116. The crown 116 is attached to the first portion of the movement 112. The crown 116 can be used to set the time of day. For example, rotating the crown 116 can cause the hands 108, 110 of the biofeedback watch to point to particular markings on the indicator ring 114. The crown 116 may need to be engaged before its rotation causes the hands 108, 110 of the biofeedback watch to rotate. The crown 116 may be engaged by pulling the crown 116 away from the case 102.

Referring again to FIG. 1, the body 100 of the biofeedback watch includes a transparent crystal 106 that is set into the case 102. The crystal 106 is a disk that creates a barrier between (i) the dial 104, the hands 108, 110, the movement 112, and the indicator ring 114 and (ii) the exterior of the case 102. The crystal 106 can be formed of any of a number of materials, such as glass, acrylic, polycarbonate, mineral glass, Gorilla Glass™, synthetic sapphire, or any other suitable material. The crystal 106 is disposed in a bezel 122, and the bezel 122 is affixed to the indicator ring 114. An L-ring (or “Glass Gasket”) made from a nylon elastomer is used to create a seal between the crystal 106 and the bezel 122, thereby protecting the biofeedback watch from water, dirt, dust, and other debris. The vertical part of the L-ring creates friction that prevents the crystal 106 from becoming dislodged from the bezel 122. The horizontal part of the L-ring creates a seal between the crystal 106 and the bezel 122.

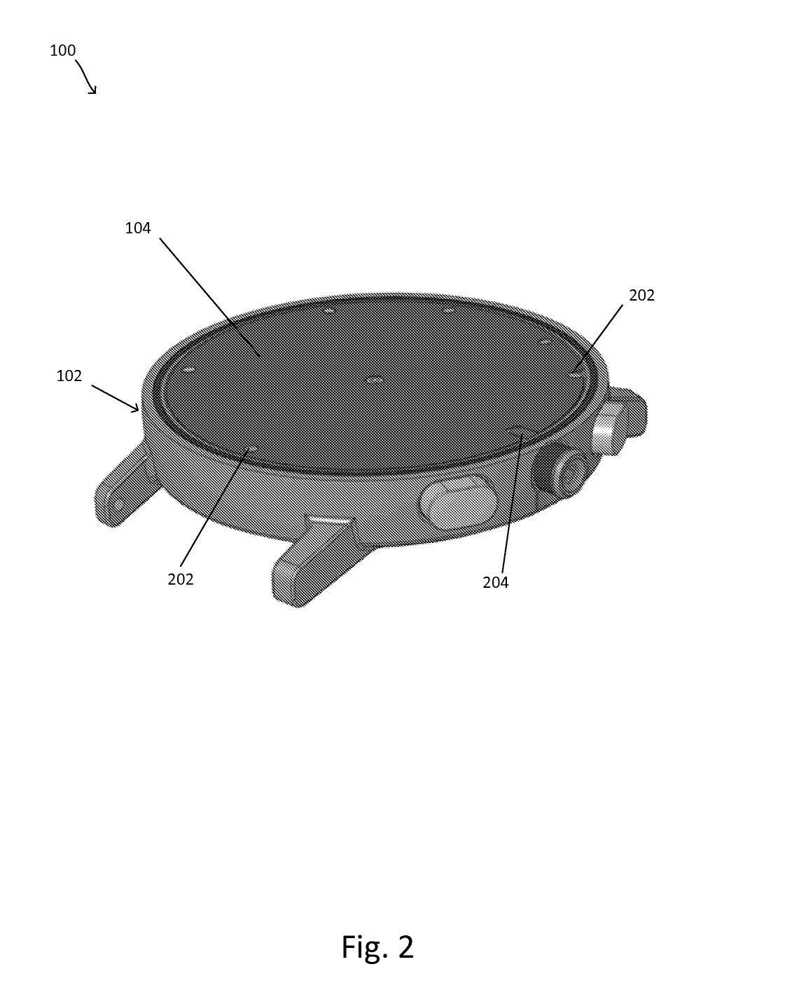

In FIG. 2, the crystal 106, indicator ring 114, movement 112, and hands 108, 110 have been removed. The dial 104 has various holes 202 and a notch 204 around its circumference. The holes 202 align with pins 308 (shown in FIG. 3) that extend into the holes, securing the dial 104 in place. The notch 204 aligns with a portion of the indicator ring 114 that extends into the notch, ensuring that the dial 104 and the indicator ring 114 are positioned correctly relative to each other.

FIG. 3 is a perspective view of the body 100 with the dial 104 removed, showing portions of a chassis 302, portions of a sub-chassis 304, two OLED displays 306a, 306b, and an antenna 312. The chassis 302 and the sub-chassis 304 reside inside the body 102 and provide a frame to which various components are connected. The chassis 302 and the sub-chassis 304 are shaped such that components fit together in a stable and compact package. The pins 308 that extend through the holes 202 of the dial 104 engage the sub-chassis 304 to hold the dial 104 in a fixed position.

The OLED displays 306a, 306b are positioned in the 12 o'clock (e.g., upper) and 6 o'clock (e.g., lower) hemispheres of the case 102 and are positioned beneath the dial 104 (shown in FIGS. 1 and 2). The chassis 302 and the sub-chassis 304 assist in holding the OLED displays 306a, 306b in place. Flexible printed circuits (FPCs) 310a, 310b are electrically connected to each of the OLED displays 306a, 306b. The FPCs 310a, 310b are ribbon cables that connect the OLED displays 306a, 306b to a printed circuit board (PCB) 604 (shown in FIG. 6), as described in more detail later.

The OLED displays 306a, 306b are high-resolution, pixelated displays that include numerous LEDs that are capable of emitting light having various colors. Because of their high resolutions, the OLED displays 306a, 306b are able to display relatively complex images. For example, unlike an LCD display, which can typically only display coarse black segments, the OLED displays 306a, 306b are able to display high-resolution pictures and symbols in color. As such, the OLED displays 306a, 306b can display information related to the biofeedback functions of the biofeedback watch.

The OLED displays 306a, 306b do not require a backlight. As such, the OLED displays 306a, 306b can display deep black levels, which can help some portions of the display remain hidden beneath the deadfront window when those portions are not active. The lack of a backlight also allows the OLED displays to be thinner and lighter than LCD displays.

Any of various different OLED displays can be used. In some implementations, the OLED displays 306a, 306b are Pioneer™ MXS4097-A OLED display units that have high pixel density and high color depth. In some implementations, each of the OLED displays 306a, 306b uses a passive-matrix addressing scheme, has a resolution of 128×32 pixels, has a thickness of 1.0 mm, has a visible area of 21.52×6.058 mm, has an active area of 20.322×5.058 mm, has a dot pitch of 0.159×0.159 mm, has a subdot pitch of 0.053×0.159 mm, has a Serial (SPI) interface, and includes an LD7138/LDT OLED driver IC.

When active, the OLED displays 306a, 306b require a relatively high amount of power to operate compared to the rest of the electrical components of the biofeedback watch. For this reason, the OLED displays 306a, 306b typically are not used for timekeeping functions of the biofeedback watch, as timekeeping functions would require the OLED displays 306a, 306b to be active for extended periods of time. Rather, the biofeedback watch keeps analog time using the movement 112 (shown in FIG. 1). Operating the movement 112 requires significantly less power than operating the OLED displays 306a, 306b.

Each of the OLED displays 306a, 306b has foam affixed to its bottom surface. The foam resides between the OLED displays 306a, 306b and the components of the biofeedback watch that reside toward the bottom portion of the case 102 (e.g., the movement 112 and the sub-chassis 304). Each piece of foam has an hourglass shape that covers most of the bottom surface of its corresponding OLED display 306a, 306b. The portions of the bottom surfaces of OLED displays 306a, 306b that are not covered with foam allow for the gears of the movement 112 to remain unobstructed. The foam is formed of a soft, compressible material that can be pressed between components of the biofeedback watch without damaging them. The foam fills voids in the case 102 to securely fix the components of the biofeedback watch in place. For example, if there is any positional variance between components of the biofeedback watch that would otherwise cause the components to shift around, the foam presses down on these components to hold them in place.

Still referring to FIG. 3, an antenna 312 is disposed next to the OLED displays 306a, 306b. The antenna 312 is an inverted F antenna that is electrically connected to a Bluetooth low energy (BLE) radio. The antenna 312 allows the BLE radio to establish Bluetooth connections with other BLE radios, thereby allowing the biofeedback watch to wirelessly communicate with BLE-enabled devices. The antenna 312 is positioned and tuned such that it can sufficiently communicate with BLE radios of other devices.

FIG. 4 is a perspective cross-sectional view of the body 100, showing a battery 402 and the first portion 902 (shown in FIG. 9) of the movement 112. The battery 402 is shaped such that it can occupy maximal unused space within the case. As such, the battery 402 can be relatively large while still fitting within the constrained dimensions of the case 102. The battery 402 may be specifically designed for use in the biofeedback watch. The battery 402 is electrically connected to a PCB 604 (shown in FIG. 6) that resides in the case 102. The PCB 604 is also electrically connected to the OLED displays 306a, 306b via the FPCs 310a, 310b (shown in FIG. 3). The battery 402 powers the PCB 604, the OLED displays 306a, 306b, and the other components that are electrically connected to the PCB 604.

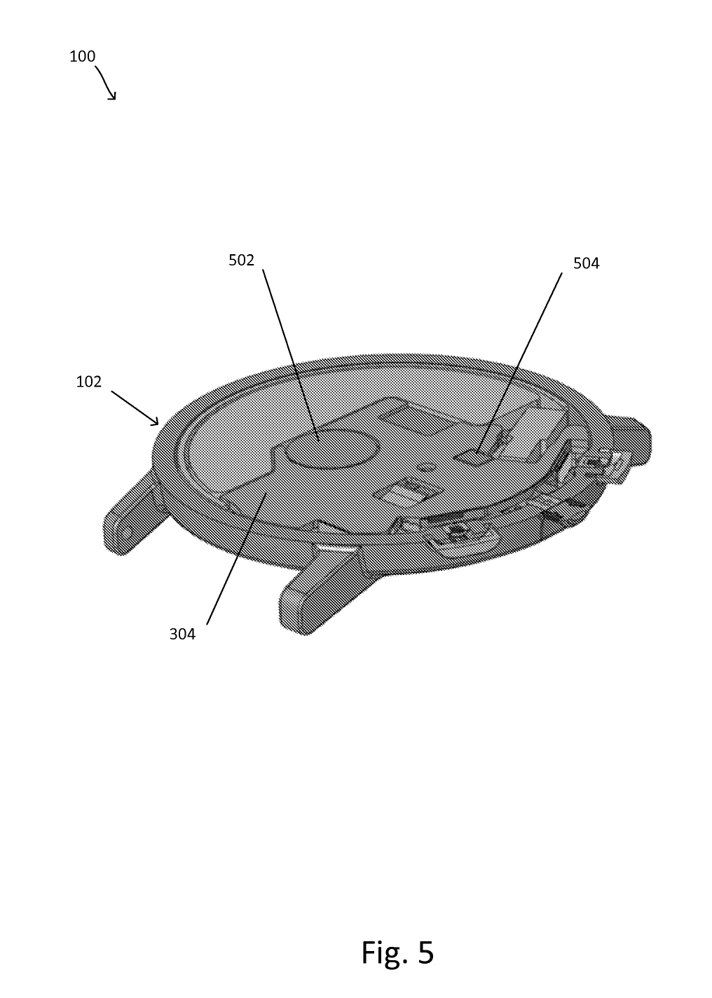

Still referring to FIG. 4, the first portion 902 of the movement 112 resides next to the battery 402 and the sub-chassis 304 substantially in the center of the case 102. The first portion 902 of the movement 112 includes a movement battery 502 (shown in FIG. 5), gears, coils, and a control board for controlling the gears.

The movement 112 also includes a rod 404 that connects the first portion 902 of the movement 112 to the crown 116. Specifically, the rod 404 can connect the gears to the crown 116. The crown 116 can be rotated, causing the gears to rotate accordingly, and thus causing the second portion 904 (shown in FIG. 9) of the movement 112 and the hands 108, 110 (shown in FIG. 1) to rotate.

The buttons 118a, 118b can communicate with the processor 608 (shown in FIG. 6) and cause the processor 608 to control the internal components of the biofeedback watch. Switches 406a, 406b are positioned such that the buttons 118a, 118b engage their respective switch 406a, 406b when the buttons 118a, 118b are pressed. The switches 406a, 406b are electrically connected to the PCB 604 (shown in FIG. 6) and the processor 608. When engaged (e.g., momentarily or as a toggle), the switches 406a, 406b facilitate an electrical connection in the PCB 604 that causes the processor 608 to initiate one or more control signals. Thus, a user can press the buttons 118a, 118b to control the functions of the biofeedback watch, as described in more detail later.

FIG. 5 is a perspective cross-sectional view of the body 100, showing the movement battery 502, which resides beneath the first portion 902 (shown in FIG. 9) of the movement 112 (shown in FIG. 4), and a vibration motor 504. The movement battery 502 can be a coin cell battery that is dedicated to powering the movement 112. The movement battery 502 is electrically connected to the control board of the movement, and the control board is connected to the gears. The movement battery 502 provides power to the control board, which causes the gears of the movement 112 to rotate. The gears are configured and arranged to interact with one another to cause the second portion 904 (shown in FIG. 9) of the movement 112 to rotate in a particular manner. The rotation causes the hands 108, 110 (shown in FIG. 1) that are connected to the second portion 904 of the movement 112 to indicate a time of day.

The vibration motor 504 is electrically connected to the PCB 604 and controlled by the processor 608 (shown in FIG. 6). The vibration motor 504 is configured to provide haptic feedback to the user. For example, the vibration motor 504 can be configured to vibrate in response to an input from the user. The vibration motor 504 can be configured to vibrate in order to alert the user that a particular event has occurred.

FIG. 6 is a perspective cross-sectional view of the body 100 showing the PCB 604 and the printed circuit board assembly (PCBA) 606, which are located beneath the sub-chassis 304 (shown in FIG. 5). The PCBA 606 includes the PCB 604 and the components that are electrically connected to the PCB 604. The PCBA 606 is affixed to the sub-chassis 304. A processor 608 is electrically connected to the PCB 604. The processor 608 is a microcontroller that is configured to execute instructions for performing the functions of the biofeedback watch. Memory modules are electrically connected to the PCB 604. The memory modules are for storing software that includes the instructions for performing the functions of the biofeedback watch.

A display connector 602 is electrically connected to the PCB 604. The display connector 602 accepts the FPCs 310a, 310b (shown in FIG. 3). Each of the FPCs 310a, 310b electrically connects its corresponding OLED display 306a, 306b (shown in FIG. 3) to the PCB 604 via the display connector 602. The processor 608 controls what is displayed on the OLED displays 306a, 306b.

Still referring to FIG. 6, a motion sensor 610 for measuring movement is electrically connected to the PCB 604. The motion sensor 610 is configured to measure MoCG data, as described above, and to provide the MoCG data to the processor 608. The motion sensor 610 includes multiple accelerometers (e.g., one for each of the x, y, and z axes). The MoCG data can be used in combination with collected PPG data to determine certain vital signs of the wearer, such as heart rate, pulse transit time, stroke volume, systolic and diastolic blood pressure, and cardiac output.

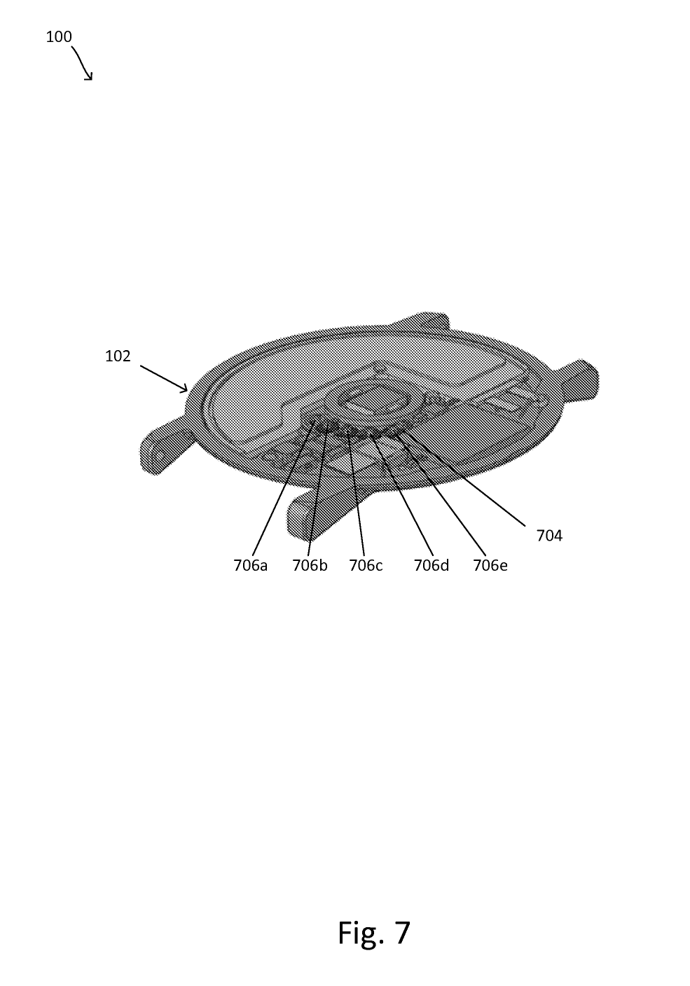

FIG. 7 is a perspective cross-sectional view of the watch body 100 showing a USB contacts housing 704 that is located beneath the PCBA 606 (shown in FIG. 6). The USB contacts housing 704 has four holes that are configured to accept four USB contacts 806b-e (shown in FIG. 8). Five contact pins 706a-e are electrically connected to the PCB 604 (shown in FIG. 6). Each contact pin 706a-e can, for example, be electrically connected to a spring that facilitates the electrical connection to the PCB 604. Four of the contact pins 706b-e have corresponding bottom USB contacts 806b-e that are exposed on the bottom wall 802 (shown in FIG. 8) of the biofeedback watch.

FIG. 8 is a bottom view of the biofeedback watch body 100 showing a sensor assembly insert 801 and the USB contacts housing 704. USB contacts 806b-e are electrically connected to four of the contact pins 706b-e (shown in FIG. 7), respectively, and are exposed on the bottom wall 802 of the biofeedback watch. The USB contacts 806b-e are disposed in the holes of the USB contacts housing 704. The interface between the USB contacts 806b-e and the USB contacts housing 704 prevents water from entering the biofeedback watch. An external USB connector can make contact with the USB contacts 806b-e, allowing an external USB device to communicate with the PCB 604. Each of the USB contacts 806b-e corresponds to a USB pin: 806b corresponds to Ground, 806c corresponds to Data+, 806d corresponds to Data −, and 806e corresponds to V_{BUS}. A USB cord can be electrically connected to the biofeedback watch via the USB contacts 806b-e. The USB cord can have a first end that includes an attachment mechanism that has four pins for making contact with the USB contacts 806b-e of the contact pins 706b-e. The USB cord can plug into a power source that is used to charge the biofeedback watch. The cord can plug into a computer, allowing the computer to communicate with the biofeedback watch.

An external sensor 808 for making contact with a user's skin is disposed in one of the holes of the USB contacts housing 704. The external sensor 808 is electrically connected to the contact pin 706a. The external sensor 808 is a capacitive sensor configured to detect whether the biofeedback watch is being worn by a user. The external sensor 808 can have a bottom surface area of approximately 20 mm². When the biofeedback watch is not being worn, as detected by the external sensor 808, one or more components of the biofeedback watch can be powered off and one or more functions of the biofeedback watch can be suspended.

FIG. 9 shows a perspective cross-sectional view of the biofeedback watch. As described above, the movement 112 has a first portion 902 and a second portion 904. The second portion 904 of the movement 112 includes a cannon 906. The hands 108, 110 are attached to the cannon 906. The cannon 906 acts as an extender for the second portion 904 of the movement 112 so that the hands 108, 110 are positioned at an appropriate height relative to the dial 104. Specifically, the cannon 906 extends through the dial 104 (e.g., the deadfront window) of the watch body 100 and extends a sufficient distance above the dial 104 to permit the hands 108, 110 to be connected to the cannon. In this way, the cannon 906 permits conventional movements designed for thinner, conventional wristwatches to be used with the watch body 100.

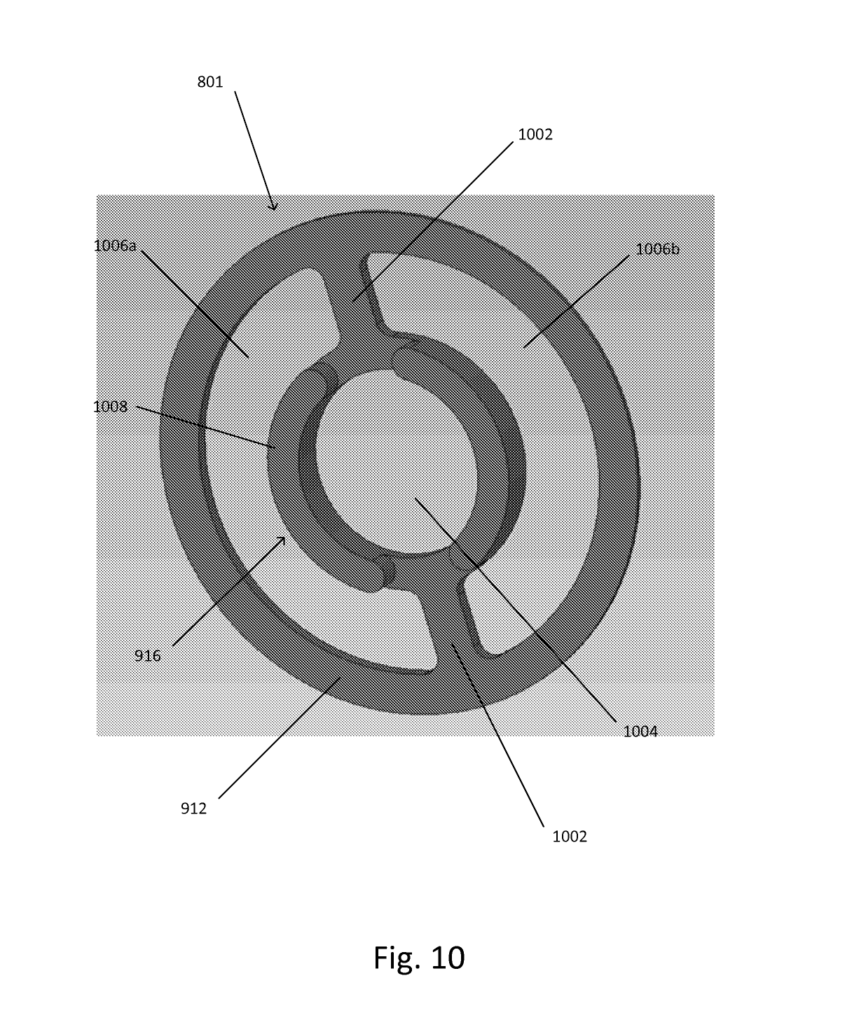

FIG. 10 shows a perspective view of the sensor assembly insert 801. Referring to FIGS. 8-10, the sensor assembly insert 801 is made of one contiguous piece of material and is configured to reside in an aperture formed by the bottom wall 802 of the case 102. The sensor assembly insert 801 is a disk that includes an inner circular wall 916 and an outer circular wall 912 connected to the inner circular wall 916 by connector segments 1002. The inner wall 916 and the outer wall 912 are concentric rings, and the segments 1002 connect the inner wall 916 to the outer wall 912 at two opposite locations. The inner wall 916 forms a circular first opening 1004 (e.g., a central opening), and the spaces between the inner wall 916 and the outer wall 912 form arc-shaped second openings 1006a, 1006b. That is, the inner wall 916 separates the first opening 1004 from the second openings 1006a, 1006b, and the segments 1002 separate the second openings 1006a, 1006b from each other. As shown in FIGS. 9 and 10, the inner wall 916 includes raised regions 1008 that partially surround an optical sensor 910. These raised regions 1008 of the inner wall 916 help to prevent light emitted by LEDs 908a, 908b from reaching the optical sensor 910 before first being reflected from a wearer's skin.

Referring now to FIGS. 8 and 9, the first opening 1004 is configured to align with the optical sensor 910 disposed within the case 102. The optical sensor 910 can be a photodiode. The optical sensor 910 is electrically connected to the PCB 604. The second openings 1006a, 1006b are configured to align with the LEDs 908a, 908b disposed within the case 102. The LEDs 908a, 908b are also electrically connected to the PCB 604.

Referring to FIG. 9, a window 914 that separates the optical sensor 910 from the exterior of the case 102 resides in the first opening 1004. The window 914 protects the biofeedback watch from water, dirt, dust, and other debris. The window 914 also provides an appropriate medium through which the optical sensor 910 can receive light. The window 914 is made of acrylic.

Lenses 918a, 918b that separate the LEDs 908a, 908b from the exterior of the case 102 reside in the second openings 1006a, 1006b. The lenses 918a, 918b protect the biofeedback watch from water, dirt, dust, and other debris. The lenses 918a, 918b can be formed of any of a number of materials, such as acrylic, glass, plastic, polycarbonate, or any other suitable material.

The LEDs 908a, 908b are configured to emit light through the lenses 918a, 918b and illuminate the skin with the light, and the optical sensor 910 is configured to measure the amount of light transmitted or reflected from the skin in order to measure PPG data in combination with the MoCG data. The PPG data is provided to the processor 608 (shown in FIG. 6). As noted above, the PPG data can be used to determine certain vital signs of the wearer, such as heart rate, pulse transit time, stroke volume, systolic and diastolic blood pressure, and cardiac output.

The inner wall 916 (e.g., the raised regions 1008 of the inner wall 916) of the sensor assembly insert 801 separates the LEDs 908a, 908b from the optical sensor 910. This arrangement helps to prevent light emitted from the LEDs 908a, 908b from entering the first opening 1004 (shown in FIG. 10), and being observed by the optical sensor 910. As a result, light is not typically detected by the optical sensor 910 until it reflects off of the skin of the user. This can increase the accuracy with which vital signs are determined. Without the inner wall 916, light from the LEDs 908a, 908b would leak into the first opening without first reflecting off of the skin of the user, leading to inaccurate readings for the optical sensor 910.

The sensor assembly insert 801 is precisely aligned in relation to the LEDs 908a, 908b and the optical sensor 910. For example, the LEDs 908a, 908b and the optical sensor 910 may be calibrated for a particular alignment with the window 914 and the lenses 918a, 918b. As such, any variance between the actual alignment and the calibrated alignment can result in inaccurate measurements by the optical sensor 910. The sensor assembly insert 801 is configured to tightly fit in the aperture formed by the bottom wall 802 of the case 102. As described above, the case 102 is formed from a single contiguous piece of material such that the bottom wall 802 of the case 102 is formed integrally with and is not removable from the rest of the case 102. The LEDs 908a, 908b and the optical sensor 910 are electrically connected to the PCB 604 (which is affixed to the sub-chassis 304 in the case 102), but the sensor assembly insert 801 resides in the bottom wall 802 of the case 102. If the bottom wall 802 of the case 102 and the rest of the case 102 were two separate pieces, as is the case in many traditional watches, an additional possible positional variance of the sensor assembly insert 801 in relation to the LEDs 908a, 908b and the optical sensor 910 would be introduced. The “monoblock” structure of the case 102 reduces the positional variance of the sensor assembly insert 801, the window 914, and the lenses 918a, 918b in relation to the LEDs 908a, 908b and the optical sensor 910. Also, because the sensor assembly insert 801 is a single component that defines the openings (which contain the window 914 and the lenses 918a, 918b), any positional variance between the openings that may otherwise result if the openings were formed by separate components is eliminated.

As described above, each of the OLED displays 306a, 306b (shown in FIG. 3) has foam affixed to its bottom surface for filling voids in the case 102 to securely fix the components of the biofeedback watch in place. In this way, the foam increases the pressure exerted from above on the sensor assembly insert 801, thereby helping to hold it securely in place.

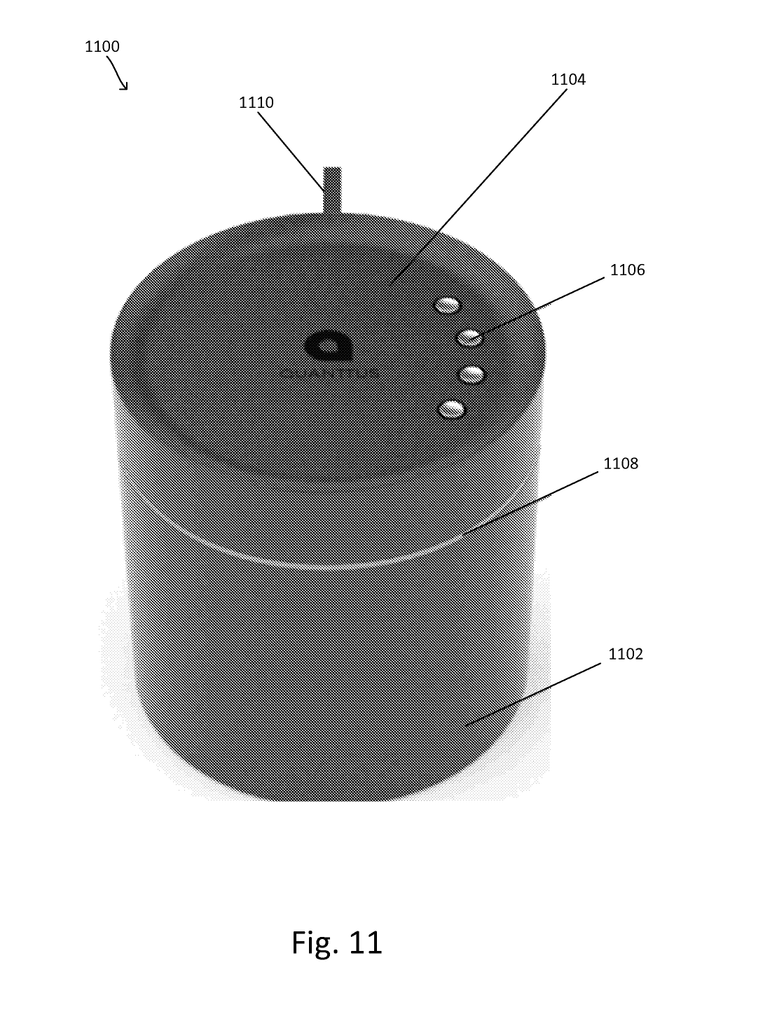

FIG. 11 shows a perspective view of a charging dock 1100 for the biofeedback watch. The dock 1100 has a base 1102, a top surface 1104 upon which the biofeedback watch can sit, electrical contacts 1106 for making electrical contact with the USB contacts 806b-e (shown in FIG. 8) of the biofeedback watch, an LED ring 1108 for indicating a charging condition, and a power cord 1110 for connecting the dock 1100 to a power source (e.g., an electrical outlet). The dock 1100 is formed of a molded rubber material. The dock 1100 includes a USB cord for connection to a computer, such that the computer and the biofeedback watch can communicate with each other.

When the biofeedback watch is placed on the top surface 1104 of the dock 1100, the electrical contacts 1106 of the dock make an electrical connection with the contacts USB 806b-e of the biofeedback watch, allowing the biofeedback watch to receive an electrical charge via the power cord 1110. The top surface 1104 of the dock 1100 is magnetic and configured to attract the body 100 of the biofeedback watch. The magnetic attraction helps the biofeedback watch sit securely in the appropriate position on the top surface 1104 so that the contacts USB 806b-e and the electrical contacts 1106 are properly aligned. The LED ring 1108 can indicate the charging condition of the biofeedback watch. When the watch is charging, the LED ring 1108 is red. When the watch is fully charged, the LED ring 1108 is green. As such, a user can determine the charge status of the biofeedback watch without interacting with the biofeedback watch itself.

FIG. 12 shows a biofeedback watch 1200 being worn by a user. The watch 1200 includes a wristband 1202 connected to the watch body 100. Like a traditional wristwatch, the biofeedback watch is typically worn by the user throughout the day. As the user goes about his or her daily activities, the biofeedback watch continuously collects data using the motion sensor 610 and the optical sensor 910. The data is collected at a frequency of 128 Hz. However, other frequencies can be used, depending on the intended use of the data. The motion sensor 610 measures MoCG data, and the optical sensor 910 measures PPG data, as described above. The data collection occurs in the background of the timekeeping functions of the biofeedback watch and does not require management by the user.

The MoCG data and the PPG data are provided to the processor 608. The processor 608 causes the MoCG data and the PPG data to be analyzed to determine biometric measurements (e.g., heart rate, pulse transit time, stroke volume, systolic and diastolic blood pressure, and cardiac output) of the user. Examples of methods of using MoCG data and PPG data to determine such biometric measurements are described in U.S. Provisional Patent Application No. 61/894,884, entitled “Consumer Biometric Devices,” and U.S. Provisional Patent Application No. 62/002,531, entitled “Consumer Biometric Devices,” each of which is incorporated by reference herein.

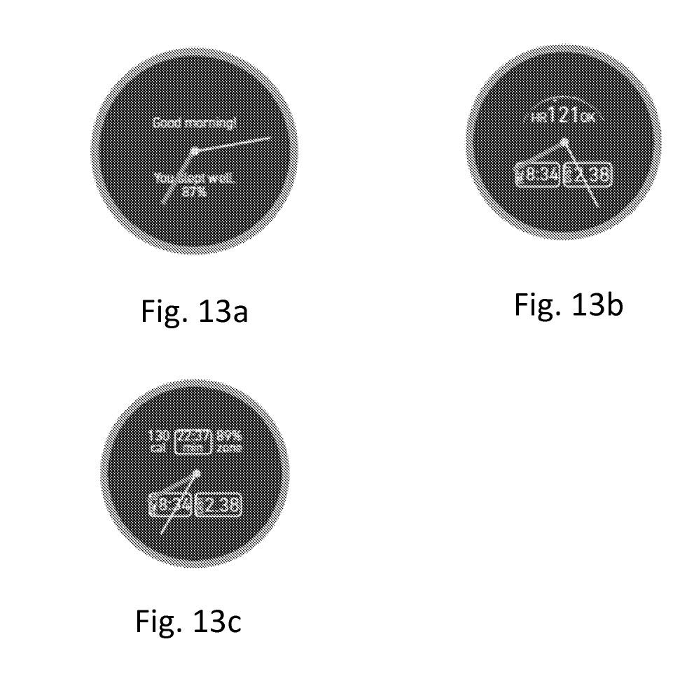

FIGS. 13a-c show a series of screenshots of the biofeedback watch 1200 during an example use of the biofeedback watch 1200.

The biofeedback watch 1200 can determine when the user is sleeping based on MoCG data and PPG data measured by the motion sensor 610 and the optical sensor 910, respectively. For example, the measured data may indicate that the user has been in a substantially stationary position for a prolonged period of time while exhibiting vital signs typically seen in someone who is asleep. While the user is sleeping, the biofeedback watch 1200 is in a partial sleep state in which less than all of the functions of the biofeedback watch 1200 operate. For example, the OLED displays 306a, 306b of the biofeedback watch 1200 are powered off while the user is sleeping. The biofeedback watch 1200 can determine the user's sleep characteristics while the user is sleeping by measuring and analyzing the MoCG data and PPG data.

Upon waking, the user presses one of the buttons 118a, 118b (shown in FIG. 1) to cause the processor 608 to fully awaken the biofeedback watch 1200. The OLED displays 306a, 306b are activated, and the user is presented with a message indicating a “sleep score” that is represented as a percentage, as shown in FIG. 13a. The user may also be presented with suggestions regarding how the user can improve his sleep.

The biofeedback watch can perform both passive and active functions. The biofeedback watch 1200 can perform a number of functions that require initiation by the user. For example, before exercising, the user can use the buttons 118a, 118b to select an exercise type (e.g., going for a run). The user can then use the buttons 118a, 118b to indicate a beginning of the run. During exercise, the biofeedback watch 1200 displays the user's heart rate, the run pace, and the current distance ran, as shown in FIG. 13b. The user can indicate that exercise is complete by pressing one of the buttons 118a or 118b. Once the exercise is complete, the biofeedback watch 1200 displays the number of calories burned by the user, the total length of the run, the average run pace, and the total distance ran, as shown in FIG. 13c.

At any point as the biofeedback watch 1200 analyzes the MoCG data and the PPG data to determine biometric measurements of the user, the biofeedback watch 1200 may automatically notify the user of particular biometric measurements without requiring user interaction via the buttons 118a, 118b. If the biofeedback watch 1200 determines a potentially dangerous biometric measurement, the OLED displays 306a, 306b may present a notification to the user that contains information related to the biometric measurement. For example, the biofeedback watch 1200 may determine that the user has a dangerously high heart rate. In response, the OLED displays 306a, 306b may present a notification that includes the user's current heart rate. The biofeedback watch 1200 can also invoke other components to help make the notification noticeable to the user. For example, the vibration motor 504 (shown in FIG. 5) can be activated. In some implementations, the OLED displays 306a, 306b can blink or flash, thereby making the notification more noticeable to the user.

While certain implementations have been described above, various other implementations are possible.

While the biofeedback watch has been described as including a case 102 that has a monoblock structure, in other implementations, the case of the biofeedback watch is formed of multiple pieces.

While we described the substantially opaque first ink pigment layer being deposited over the top surface of the dial 104 and the substantially transmissive second ink pigment layer being deposited on top of the first ink pigment layer, in some implementations, the first pigment layer is substantially transmissive and is deposited over the entire top surface of the dial, and the second pigment layer is substantially opaque and is deposited over the top surface of the dial on top of the first pigment layer except for the regions of the dial that align with the OLED displays.

While we described the crown 116 as being used to set the time on the biofeedback watch, in an alternative implementation, the crown can perform other functions. The crown can be in communication with other internal components of the biofeedback watch in addition to the movement, and can be used to perform functions associated with the internal components of the biofeedback watch. For example, the crown may operate as a button that can be pressed in to perform functions similar to those performed by the buttons of the biofeedback watch.

While the biofeedback watch body has been described as including OLED displays 306a, 306b, other types of displays can be used. For example, the biofeedback watch can include one or more LCD displays. The biofeedback watch can include one or more emissive or transmissive displays. The displays can use an active display technology. The displays can be positioned in the same locations as the OLED displays described above. Alternatively, a single display can reside beneath the dial.

While the biofeedback watch body has been described as including two OLED displays 306a, 306b, the biofeedback watch can include four OLED displays. Each OLED display can reside beneath each quadrant of the dial. Alternatively, a single OLED display can reside beneath the dial. In such a case, the movement can protrude through a hole at or near the center of the display.

While the biofeedback watch has been described as displaying analog time using the movement 112, in some implementations, the biofeedback watch displays the time digitally. For example, the biofeedback watch may not include a movement and hands, but rather, a display (e.g., an LCD display) can be used to display the time. In such a case, the dial of the biofeedback watch may be unnecessary because the display can replace the dial.

While the biofeedback watch has been described as including a motion sensor 610 that includes only an accelerometer, in some implementations, the motion sensor also includes one or more gyroscopes for measuring tilt, rotation, and yaw. The gyroscope can be configured to measure data that is used to refine the MoCG measurements. The gyroscope can also be configured to detect particular movements or positions of the watch. For example, the gyroscope can be configured to determine when the biofeedback watch is positioned at a particular angle. The processor may cause the displays to be turned on or off when the biofeedback watch is in a particular position. The gyroscope can also be configured to determine the number of steps a user takes while wearing the biofeedback watch.

While the biofeedback watch has been described as having four USB contacts 806b-e, the biofeedback watch can have any number of USB contacts. For example, the biofeedback watch can have a full ring of USB contacts that surround the sensor assembly insert. Alternatively, the USB contacts can be arranged in a straight line. The electrical contacts of the dock can be positioned to match the arrangement used on the biofeedback watch. In some implementations, the USB contacts of the biofeedback watch can be located at a different position on the watch. For example, the contacts can be positioned on the outside edge of the case opposite the buttons and the crown. Alternatively, the biofeedback watch can include a port for accepting a plug that is used to form an electrical connection with an external device in the same way that the USB contacts are used.

While the external sensor 808 has been described as having a bottom surface area of approximately 20 mm², the bottom surface of the external sensor may have any area. For example, the bottom surface of the external sensor may have an area of approximately 17 mm² or an area greater than 20 mm². In some implementations, the external sensor may be disposed inside the case of the biofeedback watch near the bottom surface of the case.

While one of the USB contacts 806b has been described as corresponding to the USB Ground pin, in some implementations, the USB contact that corresponds to the USB Ground pin is also a temperature sensor configured to measure the skin temperature of a user. The temperature sensor can be controlled by the processor.

While the second portion 904 of the movement 112 has been described as including a cannon 906 that acts as an extender, in some implementations, the second portion of the movement has a length that extends a sufficient distance above the dial to permit the hands to be connected to the second portion of the movement, eliminating the need for an extender.

While we described some functions of the biofeedback watch that require initiation by the user, in some implementations, these functions are passive functions. That is, the biofeedback watch can analyze the MoCG data and the PPG data to determine when a user is in a particular state or is performing a particular activity, and in turn automatically initiate these functions.

While we described the biofeedback watch as including particular types of sensors, in some implementations, the biofeedback watch includes additional sensors. For example, the biofeedback watch can include one or more electric impedance sensors (including Galvanic skin resistance sensors), hydration level sensors, skin reflection index sensors, and strain sensors that can be used in performing one or more of the measurements described above.

In some implementations, one of the additional sensors included in the biofeedback watch is an ambient noise microphone. The ambient noise microphone can be electrically connected to the PCB and controlled by the processor. The ambient noise microphone can be used to receive audio input (e.g., from the user). The ambient noise microphone can also be used to detect other ambient noise. The ambient noise measurements can be correlated with the user's vital signs. For example, the biofeedback watch can determine that particular ambient noise levels lead to particular reactions (e.g., stress levels) from the user.

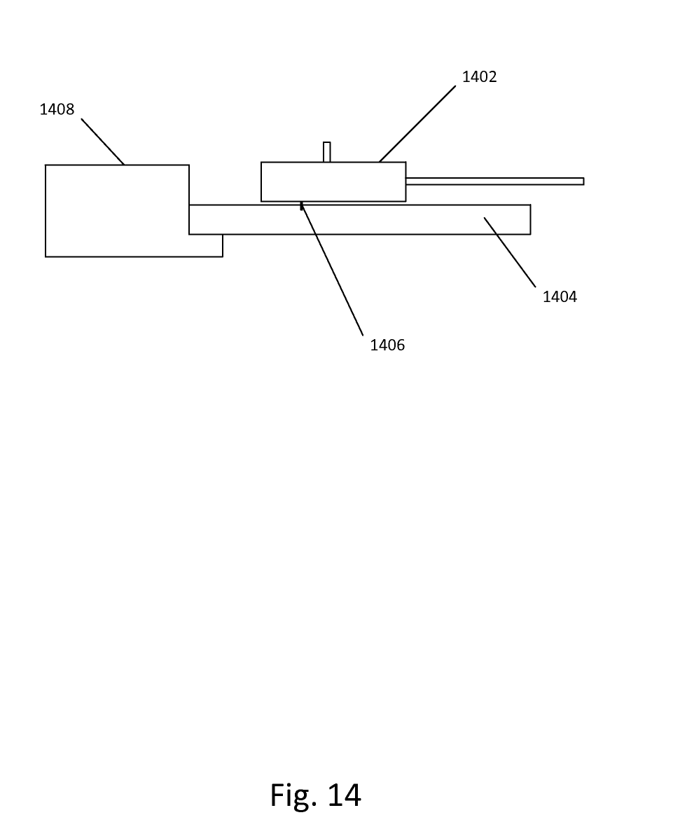

While the movement 112 has been described as being electrically connected to a movement battery 502, the movement can alternatively be connected to a different power source. FIG. 14 shows portions of an alternative implementation of the biofeedback watch with the movement 1402 electrically connected to the PCB 1404. While we have not described the other components of this implementation of the biofeedback watch in detail, it should be understood that this implementation of the biofeedback watch includes the components that were described above with reference to the body 100 (shown in FIGS. 1-9) of the biofeedback watch. In this example, the movement 1402 does not have its own dedicated movement battery. Rather, the biofeedback watch has a single power source. The movement 1402 is powered by the primary watch battery 1408. That is, the movement 1402 is powered by the same battery 1408 that powers the PCB 1404 and the components that are electrically connected to the PCB 1404 (e.g., OLED displays, a motion sensor, LEDs, an optical sensor). The movement 1402 is electrically connected to the PCB 1404 via a contact spring 1406, and the PCB 1404 is electrically connected to the battery 1408. In some implementations, instead of a contact spring 1406, some other mechanism can be used to electrically connect the movement 1402 to the PCB 1404. For example, a pin, wire trace, or a wire can electrically connect the movement 1402 to the PCB 1404. Referring to FIG. 4, the battery 1408 in the biofeedback watch takes up a substantial amount of space in the case. As described above, the battery 1408 is shaped and positioned such that it can occupy maximal unused space within the case. The battery 1408 can be set adjacent to the movement 1402 such that the battery 1408 and the movement 1402 are not stacked. Having only one battery 1408 in the biofeedback watch allows the case to have a thinner profile, giving the biofeedback watch the appearance of a traditional analog watch.

The movement 1402 is especially designed so that it can be powered by the primary watch battery 1408. While many traditional movements are driven by coils, the movement 1402 is configured to be driven electrically. Also, the movement 1402 is controlled by the processor which is electrically connected to the PCB 1404. As such, the movement 112 does not require its own dedicated control board. Eliminating the coils and the control board allows the movement 1402 to have a thinner profile and an overall smaller size, which in turn displaces fewer of the other components of the biofeedback watch. As a result, the biofeedback watch can have a maximum thickness, as measured from the bottom surface of the case to the top surface of the bezel of less than 8.80 mm and a diameter of less than 38.0 mm.

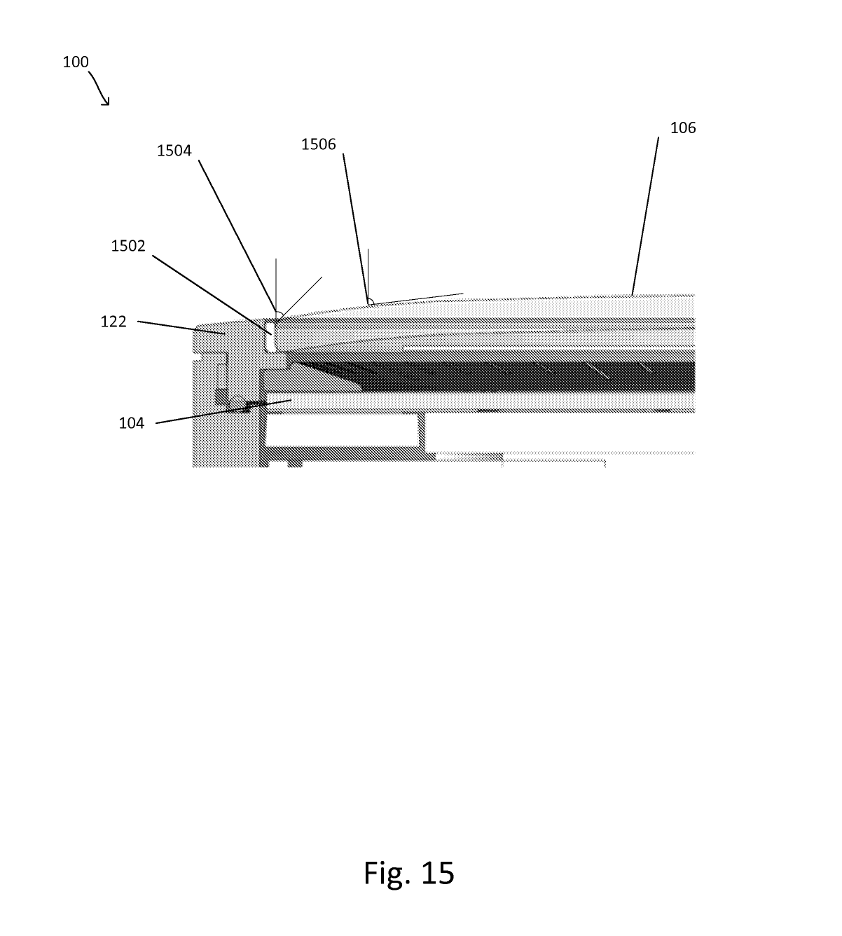

While an L-ring has been described as creating a seal between the crystal 106 and the bezel 122, other types of seals can be used. FIG. 15 shows portions of an alternative implementation of the biofeedback watch in which an I-ring 1502 creates the seal between the crystal 106 and the bezel 122. While we have not described the other components of this implementation of the biofeedback watch in detail, it should be understood that this implementation of the biofeedback watch includes the components that were described above with reference to the body 100 (shown in FIGS. 1-9) of the biofeedback watch. The I-ring 1502 can compress and create friction that prevents the crystal 1504 from becoming dislodged from the bezel 1506. An adhesive may also be applied between the I-ring 1502 and the crystal 106 or the I-ring 1502 and the bezel 122 to create a better seal.

Still referring to FIG. 15, in some implementations, the crystal 106 can be shaped such that it has a relatively steep angle of incline 1504 at the edge near the bezel 122 and a relatively shallow angle of incline 1506 towards the middle of the crystal 106. This shape for the crystal 106 can provide additional room between the crystal 106 and the dial 104 of the biofeedback watch.

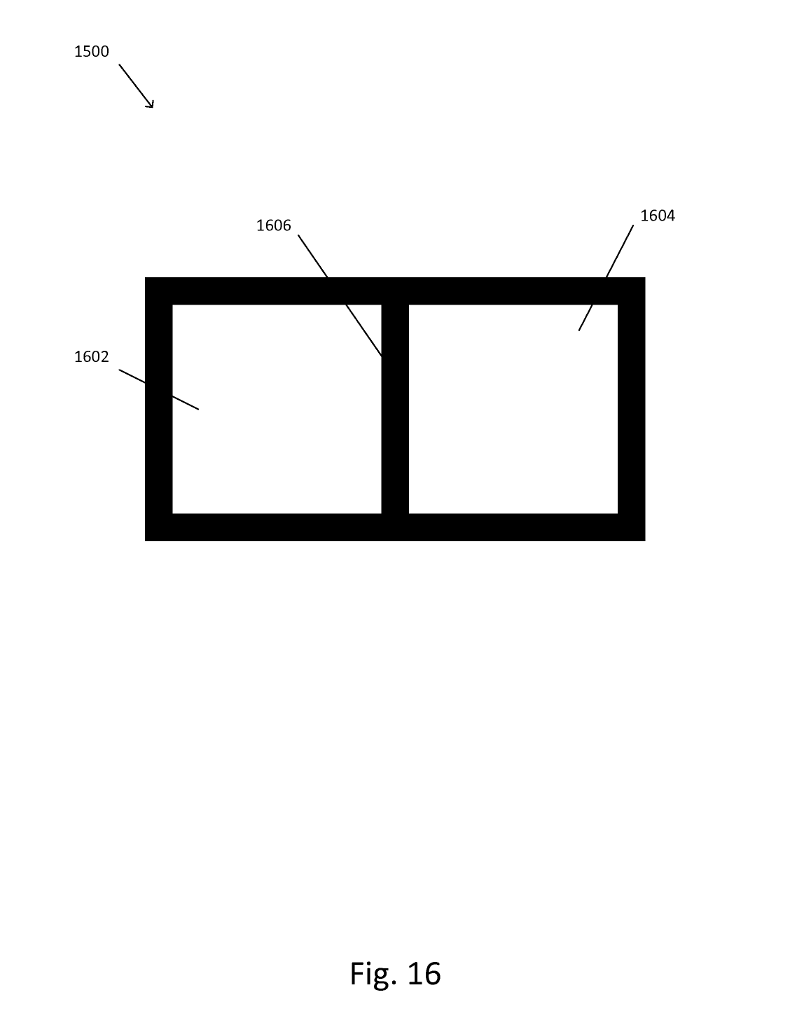

While the body 100 of the biofeedback watch body 102 has been described as including a certain number of OLED displays, LEDs, optical sensors, and motions sensors, the biofeedback watch can have any number of OLED displays, LEDs, optical sensors, and motion sensors. In some implementations, the biofeedback watch can have a single optical sensor and a single LED. Referring to FIG. 16, the sensor assembly insert 1600 can have a first opening 1602 that aligns with the optical sensor, a second opening 1604 that aligns with the LED, and a wall 1606 that separates the first opening from the second opening to prevent light emitted by the LED from reaching the optical sensor before first being reflected from a wearer's skin. The wall 1606 can have raised regions to assist in preventing light emitted by the LED from reaching the optical sensor before first being reflected from a wearer's skin.

While we described the sensor assembly insert 801 as having a circular first opening 1004 and arc-shaped second openings 1006a, 1006b, the openings in the sensor assembly insert can have any of various different shapes that permit light to pass therethrough. For example, still referring to FIG. 16, the sensor assembly insert 1600 may be rectangle shaped and have a wall 1606 separating the two halves of the rectangle to form a first square opening 1602 and a second square opening 1604. In some implementations, the openings that align with the LEDs can be circular.

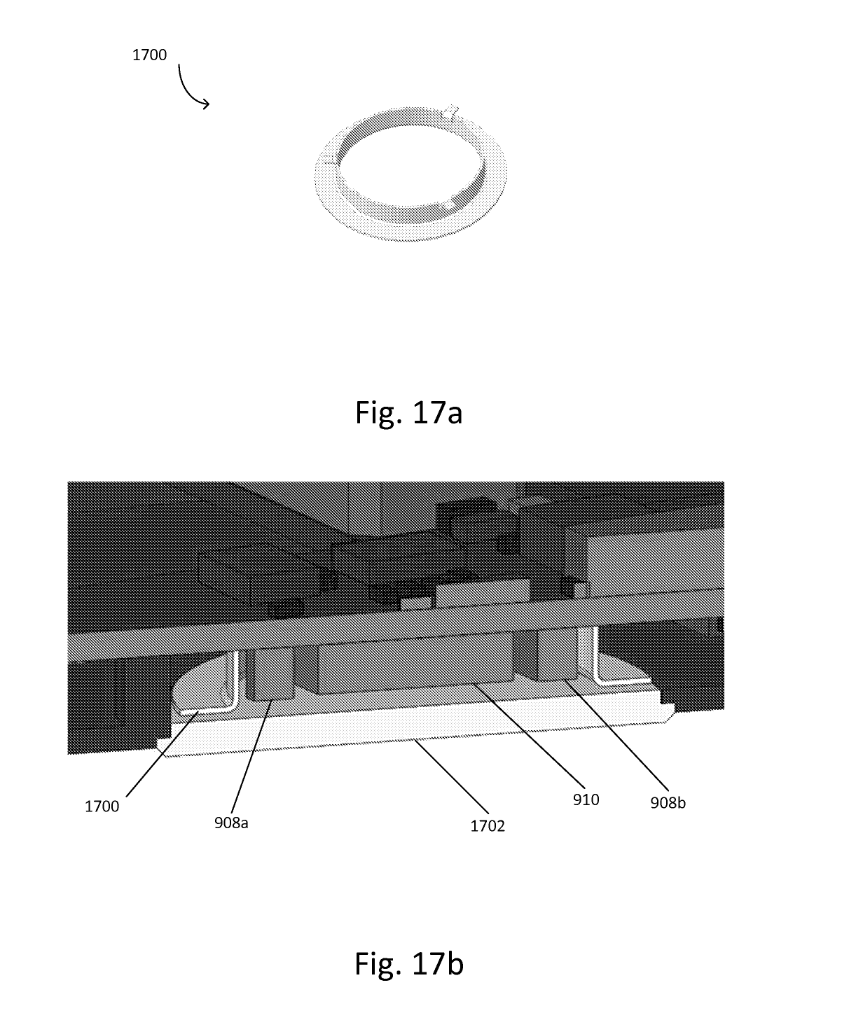

Referring to FIGS. 17a and 17b, in an alternative implementation of the biofeedback watch, the external sensor 1700 is a ring that is disposed inside the case of the biofeedback watch. While we have not described the other components of this implementation of the biofeedback watch in detail, it should be understood that this implementation of the biofeedback watch includes the components that were described above with reference to the body 100 (shown in FIGS. 1-9) of the biofeedback watch. The external sensor 1700 is disposed near the bottom surface of the case 102 and surrounds the LEDs 908a, 908b and the optical sensor 910. The external sensor 1700 is disposed on or near a window 1702 that separates the LEDs 908a, 908b and the optical sensor 910 from the exterior of the case. The external sensor 1700 is a capacitive sensor configured to detect whether the biofeedback watch is being worn by a user. A bottom surface of the external sensor 1700 has an area of approximately 22 mm². The thickness of the window 1702 is such that the external sensor 1700 can effectively measure capacitance. The sensor assembly insert is not shown in FIG. 17b.

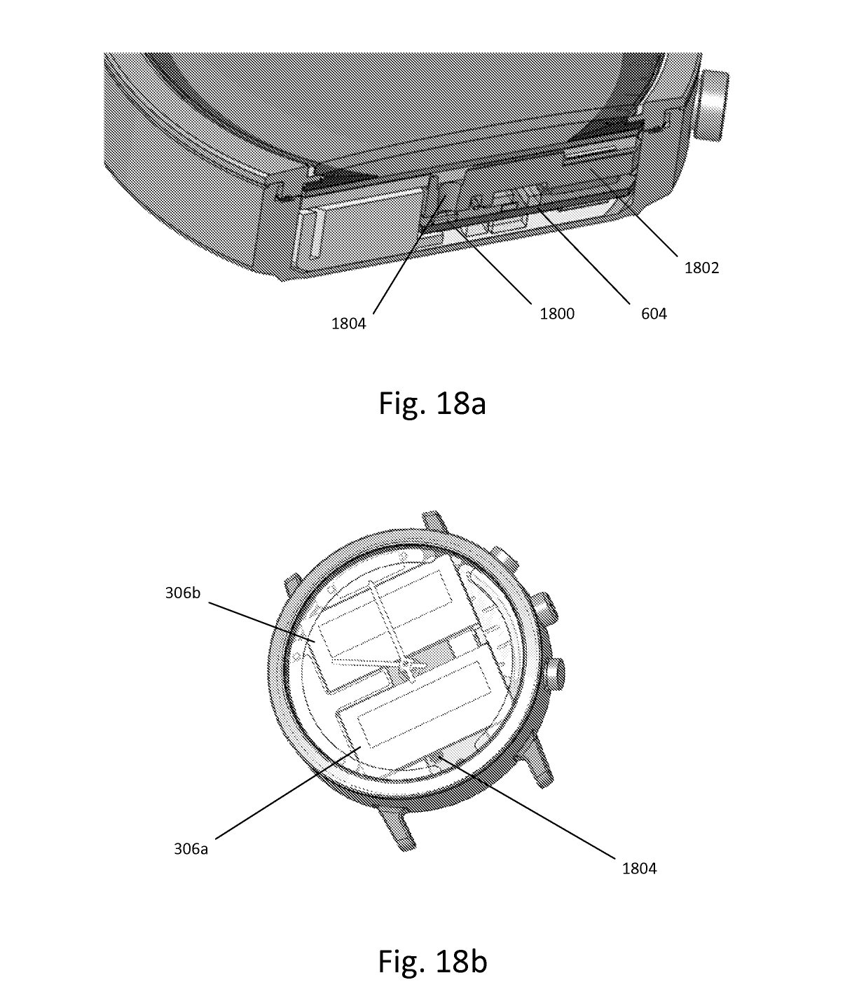

Referring to FIGS. 18a-b, an alternative implementation of the biofeedback watch includes an ambient light sensor (ALS) and ultraviolet (UV) sensor 1800. While we have not described the other components of this implementation of the biofeedback watch in detail, it should be understood that this implementation of the biofeedback watch includes the components that were described above with reference to the body 100 (shown in FIGS. 1-9) of the biofeedback watch.

The ALS and UV sensor 1800 is controlled by the processor and is electrically connected to the PCB 604 which resides beneath a sub-chassis 1802 and the dial. The sub-chassis 1802 defines a void 1804 that aligns with the ALS and UV sensor 1800. Ambient and UV light passes through the deadfront window, through the void 1804, and reaches the ALS and UV sensor 1800. Referring to FIG. 18b, the void 1804 is positioned in the sub-chassis 1802 such that the OLED displays does not obstruct light from reaching the ALS and UV sensor 1800.

The ALS components of the ALS and UV sensor 1800 measure levels of ambient light. The ambient light measurements are used to determine an appropriate brightness for the OLED displays. For example, if the user is outside on a sunny day, the ALS and UV sensor 1800 measures a high amount of ambient light. In response, the processor causes the brightness of the OLED displays to be increased for easy viewing in the environment. In contrast, if the user is in a dark room, the ALS and UV sensor 1800 measures a low amount of ambient light, and the processor causes the brightness of the OLED displays to be decreased, thereby saving battery power.

The UV components of the ALS and UV sensor 1800 measures levels UV light. The UV light measurements are used to determine the amount and intensity of UV light that the user is exposed to and the amount of time that the user spends outside. In some implementations, the ALS and the UV sensor 1800 are separate sensors.

Other implementations are within the scope of the following claims.

What is claimed is:

1. A watch comprising:
a deadfront window;
a first OLED display disposed beneath the deadfront window such that the first OLED display can be observed through the deadfront window when the first OLED display is active;
a movement for tracking time, the movement having a first portion that resides beneath the deadfront window and a second portion that protrudes through the deadfront window; and
time-indicating members disposed above the deadfront window, the time indicating members being secured to the second portion of the movement.

2. The watch of claim 1, wherein the deadfront window is a semi-transparent window that allows light to pass through when a nearby light source is active, and wherein the deadfront window is substantially opaque in the absence of a nearby active light source.

3. The watch of claim 1, wherein the watch is a biofeedback watch.

4. The watch of claim 1, wherein the first OLED display is hidden beneath the deadfront window when the first OLED display is inactive.

5. The watch of claim 1, further comprising a second OLED display, wherein the first OLED display resides beneath a first portion of the deadfront window, and the second OLED display resides beneath a second portion of the deadfront window.

6. The watch of claim 1, wherein the portion of the movement that protrudes through the deadfront window comprises an extender that is secured to a post of the movement.

7. The watch of claim 1, wherein the first OLED display has a resolution of 128×32 pixels.

8. The watch of claim 1, wherein the first OLED display is configured to emit colored light.

9. The watch of claim 1, wherein the first OLED display is a passive-matrix OLED display.

10. The watch of claim 1, further comprising a processor that is electrically connected to the first OLED display, the processor configured to:
generate data related to one or more biometric measurements; and
cause the first OLED display to display information related to one or more of the biometric measurements.

11. The watch of claim 10, wherein the biometric measurements comprise one or more of heart rate, pulse transit time, stroke volume, systolic and diastolic blood pressure, and cardiac output.

12. The watch of claim 1, wherein the first OLED display does not display a time of day.

13. The watch of claim 12, further comprising a case that contains the first OLED display and the movement.

14. The watch of claim 13, wherein the watch has a maximum thickness, as measured from a bottom surface of the case to a top surface of a bezel that is secured to the case, of less than 8.80 mm.

15. The watch of claim 13, wherein the case has a diameter of less than 38 mm.

16. The watch of claim 13, further comprising an optical sensor and an LED that are each disposed within the case.

17. The watch of claim 15, further comprising an insert disposed in an aperture formed by a bottom wall of the case, the insert having a first opening aligned with the optical sensor and a second opening aligned with the LED.

18. The watch of claim 13, wherein the case is a one-piece case.

19. The watch of claim 10, further comprising a printed circuit board electrically connected to the first OLED display and the movement, the printed circuit board being configured to provide power to the first OLED display, the movement, and the processor.

20. The watch of claim 19, wherein the printed circuit board is configured to provide power to the movement via a contact spring.

21. The watch of claim 19, wherein the printed circuit board is electrically connected to a watch battery.

22. The watch of claim 19, wherein the watch has a single power source.

23. The watch of claim 22, wherein the single power source is not directly connected to the movement.

24. A watch comprising:
a one-piece case having a bottom wall and an outer wall extending from a circumferential region of the bottom wall, the bottom wall forming an aperture;
a dial secured to the case;
a movement for tracking time, the movement having a first portion that resides beneath the dial and a second portion that protrudes through the dial;
time-indicating members disposed above the dial, the time indicating members being secured to the second portion of the movement; and
an insert disposed in the aperture formed by the bottom wall of the one-piece case, the insert having a first opening aligned with an optical sensor disposed within the case, a second opening aligned with an LED disposed within the case, and a wall that separates the first opening from the second opening.

25. The watch of claim 24, wherein the watch is a biofeedback watch.

26. The watch of claim 24, further comprising a window that resides in the first opening and a lens that resides in the second opening.

27. The watch of claim 24, wherein the insert is made of one contiguous piece of material.

28. The watch of claim 24, wherein the insert further has a third opening configured to align with a second LED disposed within the case, and wherein the wall separates the first opening from the third opening.

29. The watch of claim 28, wherein the wall is a first ring-shaped member.

30. The watch of claim 29, wherein the insert further has a second ring-shaped member concentrically disposed around the first ring-shaped member.

31. The watch of claim 30, wherein the first ring-shaped member defines the first opening and the first and second ring-shaped members cooperate to define the second and third openings.

32. The watch of claim 31, wherein the insert further has segments that extend between the first and second ring-shaped members and separate the second opening from the third opening.

33. The watch of claim 24, wherein the wall prevents light emitted from the LED from reaching the optical sensor until the emitted light passes through the second opening.

34. The watch of claim 24, further comprising:
a display disposed beneath the dial;
a processor that is electrically connected to the display, the processor configured to:
generate data related to one or more biometric measurements; and
cause the display to display information related to one or more of the biometric measurements; and
a printed circuit board electrically connected to the display, the processor, and the movement, the printed circuit board being configured to provide power to the display, the processor, and the movement.

35. The watch of claim 34, wherein the biometric measurements comprise one or more of heart rate, pulse transit time, stroke volume, systolic and diastolic blood pressure, and cardiac output.

36. The watch of claim 34, wherein the printed circuit board is configured to provide power to the movement via a contact spring.

37. The watch of claim 34, wherein the printed circuit board is electrically connected to a watch battery.

38. The watch of claim 34, wherein the watch has a single power source.

39. The watch of claim 38, wherein the single power source is not directly connected to the movement.

40. A watch comprising:
a dial;
a movement for tracking time, the movement having a first portion that resides beneath the dial and a second portion that protrudes through the dial;
time-indicating members disposed above the dial, the time indicating members being secured to the second portion of the movement;
a display disposed beneath the dial; and
a printed circuit board electrically connected to the display and the movement, the printed circuit board being configured to provide power to the display and the movement.

41. The watch of claim 40, wherein the watch is a biofeedback watch.

42. The watch of claim 40, further comprising a processor that is electrically connected to the printed circuit board and the display, the processor configured to:
generate data related to one or more biometric measurements; and
cause the display to display information related to one or more of the biometric measurements.

43. The watch of claim 42, wherein the biometric measurements comprise heart rate, pulse transit time and stroke volume, systolic and diastolic blood pressure, and cardiac output.

44. The watch of claim 42, wherein the printed circuit board is configured to provide power to the processor.

45. The watch of claim 40, wherein the printed circuit board is configured to provide power to the movement via a contact spring.

46. The watch of claim 40, wherein the printed circuit board is electrically connected to the watch battery.

47. The watch of claim 40, wherein the watch has a maximum thickness, as measured from a bottom surface of a case of the watch to a top surface of a bezel that is secured to the case, of 8.80 mm.

48. The watch of claim 40, wherein a case of the watch has a diameter of less than 38 mm.

49. The watch of claim 45, wherein the watch has a single power source.

50. The watch of claim 49, wherein the single power source is electrically connected to the printed circuit board via a first connection and the movement is connected to the printed circuit board via the contact spring.

51. The watch of claim 49, wherein the single power source is positioned adjacent to the movement.

52. The watch of claim 49, further comprising an optical sensor and an LED, wherein the single power source is configured to provide power to the printed circuit board, the processor, the display, the movement, the optical sensor, and the LED.

53. The watch of claim 40, wherein the display does not display a time of day.
